(19) United States
(12) Patent Application Publication
(10) Pub. No.: US 2007/0160580 A1
(43) Pub. Date: Jul. 12, 2007

(54) Interferon gamma therapies for idiopathic pulmonary fibrosis

(76) Inventor: Williamson Z. Bradford, Brisbane, CA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/556,487

(22) PCT Filed: May 13, 2004
(86) PCT No.: PCT/US04/15184

§ 371 Date: Nov. 15, 2006

Related U.S. Application Data
(60) Provisional application No. 60/471,862, filed on May 19, 2003.
(60) Provisional application No. 60/554,774, filed on Mar. 19, 2004.

(51) Int. Cl. A61K 38/21

(52) U.S. Cl. 424/85.6

(57) ABSTRACT

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-&ggr, to an individual with IPF.

### CROSS-REFERENCE

This application claims the benefit of U.S. Provisional Patent Application Nos. 60/471,862, filed May 19, 2003; and Ser. No. 60/554,774, filed Mar. 19, 2004, which applications are incorporated herein by reference in their entirety.

### FIELD OF X INVENTION

This invention is in the field of therapy of treating idiopathic pulmonary fibrosis.

### BACKGROUND OF THE INVENTION

Pulmonary fibrosis can be caused by a number of different conditions, including sarcoidosis, hypersensitivity pneumonitis, collagen vascular disease, and inhalant exposure. The diagnosis of these conditions can usually be made by careful history, physical examination, chest radiography, including a high resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

The logic of dividing idiopathic interstitial pneumonias into these categories is based not only on histology, but also on the different response to therapy and prognosis for these different entities. DIP is associated with smoking and the prognosis is good, with more than 70% of these patients responding to treatment with corticosteroids. NSIP patients are also frequently responsive to steroids and prognosis is good, with 50% of patients surviving to 15 years. In contrast, the UIP histologic pattern is associated with a poor response to therapy and a poor prognosis, with survival of only 3-5 years.

Idiopathic pulmonary fibrosis (IPF) is the most common form of idiopathic interstitial pneumonia and is characterized by the UIP pattern on histology. IPF has an insidious onset, but once symptoms appear, there is a relentless deterioration of pulmonary function and death within 3-5 years after diagnosis. The mean age of onset is 60-65 and males are affected approximately twice as often as females. Prevalence estimates are 13.2-20.2 per 100,000. The annual incidence is estimated to be 7.410.7 per 100,000 new cases per year.

Published evidence suggests that less than 20% of patients with IPF respond to steroids. In patients who have failed treatment with steroids, cytotoxic drugs such as azathioprine or cyclophosphamide are sometimes added to the steroid treatment. However, a large number of studies have shown little or no benefit of these drugs. There are currently no drugs approved for treatment of IPF.

There is a need in the art for methods of treating IPF. The present invention addresses this need.

Literature

WO 01/34180; Ziesche et al. (1999) N. Engl. J. Med 341:1264-1269; du Bois (1999) N. Engl. J. Med 341:1302-1304; U.S. Pat. No. 6,294,350; EP 795,332; King (2000) N. Engl. J. Med. 342:974-975; Ziesche and Block (2000) Wien. Klin Wochenschr. 112:785-790; Stem et al. (2001) Chest 120:213-219; Gay et al. (1998) Am. J. Respir. Crit. Care Med 157:1063-1072; Dayton et al. (1993) Chest 103:69-73.

### SUMMARY OF THE INVENTION

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ to an individual with IPF.

### FEATURES OF THE INVENTION

In one aspect, the invention features a method of treating a patient suffering from idiopathic pulmonary fibrosis (IPF), comprising administering to the patient an effective amount of IFN-γ, where the patient has a forced vital capacity (FVC) that is at least about 55% of the predicted normal value and/or has a carbon monoxide diffusing capacity (DL_{CO}) that is at least about 35% of the predicted normal value.

In another aspect, the invention features a method of treating a patient suffering from idiopathic pulmonary fibrosis, comprising administering to the patient an effective amount of IFN-γ, where the patient has an FVC that is at least about 55% of the predicted normal value and/or has a DL_{CO} that is at least about 30% of the predicted normal value.

In another aspect, the invention features a method of increasing the probability of survival of a patient suffering from IPF, comprising administering to the patient an effective amount of IFN-γ, where the patient has a forced vital capacity (FVC) that is at least about 55% of the predicted normal value and/or has a carbon monoxide diffusing capacity (DL_{CO}) that is at least about 35% of the predicted normal value.

In another aspect, the invention features a method of increasing the probability of survival of a patient suffering from IPF, comprising administering to the patient an effective amount of IFNIY, where the patient has an FVC that is at least about 55% of the predicted normal value and/or has a DL_{CO} that is at least about 30% of the predicted normal value.

In another aspect, the invention features a method of reducing the risk of death of a patient suffering from idiopathic pulmonary fibrosis (IPF), comprising administering to the patient an effective amount of IFN-γ, where the patient has a forced vital capacity (FVC) that is at least about 55% of the predicted normal value and/or has a carbon monoxide diffusing capacity (DL_{CO}) that is at least about 35% of the predicted normal value.

In another aspect, the invention features a method of reducing the risk of death of a patient suffering from IPF, comprising administering to the patient an effective amount of IFN-γ, where the patient has an FVC that is at least about 55% of the predicted normal value and/or has a DL_{CO} that is at least about 30% of the predicted normal value.

In another aspect, the invention features a method of treating a patient suffering from idiopathic pulmonary fibrosis (IPF), comprising the steps of (a) ascertaining that the patient has a forced vital capacity (FVC) that is at least about 55% of the predicted normal value and/or has a carbon monoxide diffusing capacity (DL_{CO}) that is at least about 35% of the predicted normal value and (b) administering to the patient an effective amount of IFN-γ.

In another aspect, the invention features a method of treating a patient suffering from IPF, comprising the steps of (a) ascertaining that the patient has an FVC that is at least about 55% of the predicted normal value and/or has a DL_{CO} that is at least about 30% of the predicted normal value and (b) administering to the patient an effective amount of IFNiY.

In another aspect, the invention features any of the above-described methods for treating a patient suffering from idiopathic pulmonary fibrosis in which IFN-γ is administered to the patient for a period of about 48 weeks. In other embodiments, IFN-γ is administered to the patient for a period of about 60 weeks. In yet other embodiments, IFN-γ is administered to the patient for a period of about one year. In still other embodiments, IFN-γ is administered to the patient for a period of at least about 70 weeks. In additional embodiments, IFN-γ is administered to the patient for a period of at least about 93 weeks. In further embodiments, IFN-γ is administered to the patient for a period of at least about 2 years. In still further embodiments, IFN-γ is administered to the patient for the remainder of the patient's life.

In another aspect, the invention features a method of treating a patient suffering from idiopathic pulmonary fibrosis (IPF), comprising administering to the patient an amount of IFN-γ effective to reduce the aggregate length of hospital stays due to IPF disease event-related hospital admissions experienced by the patient.

In another aspect, the invention features a method of treating a patient suffering from idiopathic pulmonary fibrosis (IPF), comprising administering to the patient an amount of IFN-γ effective to reduce the aggregate length of hospital stays due to IPF disease event-related hospital admissions experienced by the patient, where the patient has a forced vital capacity (FVC) that is at least about 55% of the predicted normal value.

In another aspect, the invention features a method of treating a patient suffering from idiopathic pulmonary fibrosis (IPF), comprising administering to the patient an amount of IFN-γ effective to reduce the aggregate length of hospital stays due to IPF disease event-related hospital admissions experienced by the patient, where the patient has a forced vital capacity (FVC) that is at least about 55% of the predicted normal value and/or has a carbon monoxide diffsing capacity (DL_{CO}) that is at least about 35% of the predicted normal value.

In another aspect, the invention features a method of treating a patient suffering from idiopathic pulmonary fibrosis (IPF), comprising administering to the patient an amount of IFN-γ effective to reduce the aggregate length of hospital stays due to IPF disease event-related hospital admissions experienced by the patient, where the patient has a forced vital capacity (FVC) that is at least about 55% of the predicted normal value and/or has a carbon monoxide diffusing capacity (DL_{CO}) that is at least about 30% of the predicted normal value.

In another aspect, the invention features any of the above-described methods of treating a patient suffering from IPF, to reduce the aggregate length of hospital stays due to IPF disease event-related hospital admissions experienced by the patient, where the IPF disease event-related hospital admissions are admissions based at least in part upon a respiratory event. In some embodiments, the respiratory event is a respiratory infection.

In another aspect, the invention features any of the above-described methods of treating a patient suffering from IPF, to reduce the aggregate length of hospital stays due to IPF disease event-related hospital admissions experienced by the patient, in which IFN-γ is administered to the patient for a period of about 48 weeks. In yet other embodiments, IFN-γ is administered for a period of about 60 weeks. In other embodiments, IFN-γ is administered to the patient for a period of about one year. In still other embodiments, IFN-γ is administered to the patient for a period of at least about 70 weeks. In additional embodiments, IFN-γ is administered to the patient for a period of at least about 93 weeks. In further embodiments, IFN-γ is administered to the patient for a period of at least about 2 years. In still further embodiments, IFN-γ is administered to the patient for the remainder of the patient's life.

In another aspect, the invention features kits and articles of manufacture. In some embodiments, the kit or article of manufacture comprises: (a) a container comprising an amount of IFN-γ for the treatment of a patient suffering from idiopathic pulmonary fibrosis (IPF); and (b) a label comprising printed instructions for the administration to the patient of the amount of IFN-γ in order to effect the clinical outcome of a reduction in the aggregate length of hospital stays due to IPF disease event-related hospital admissions experienced by the patient.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration to the patient of the amount of IFN-γ in order to effect the clinical outcome of a reduction in the aggregate length of hospital stays due to IPF disease event-related hospital admissions experienced by the patient, where the patient has a forced vital capacity (FVC) that is at least about 55% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration to the patient of the amount of IFN-γ in order to effect the clinical outcome of a reduction in the aggregate length of hospital stays due to IPF disease event-related hospital admissions experienced by the patient, where the patient has a forced vital capacity (FVC) that is at least about 55% of the predicted normal value and/or has a carbon monoxide difffusing capacity (DL_{CO}) that is at least about 35% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration to the patient of the amount of IFN-γ in order to effect the clinical outcome of a reduction in the aggregate length of hospital stays due to IPF disease event-related hospital admissions experienced by the patient, where the patient has a forced vital capacity (FVC) that is at least about 55% of the predicted normal value and/or has a carbon monoxide difflusing capacity (DL_{CO}) that is at least about 30% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration of the amount of IFN-γ to the patient, where the patient has a forced vital capacity (FVC) that is at least about 55% of the predicted normal value and/or has a carbon monoxide diffusing capacity (DL_{CO}) that is at least about 35% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration of the amount of IFN-γ to the patient, where the patient has a forced vital capacity (FVC) that is at least about 55% of the predicted normal value and/or has a carbon monoxide diffusing capacity (DL_{CO}) that is at least about 30% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration of the amount of IFN-γ to the patient in order to effect the clinical outcome of an increase in the probability of survival of the patient, where the patient has a forced vital capacity (FVC) that is at least about 55% of the predicted normal value and/or has a carbon monoxide diffusing capacity (DL_{CO}) that is at least about 35% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration of the amount of IFN-γ to the patient in order to effect the clinical outcome of an increase in the probability of survival of the patient, where the patient has a forced vital capacity (FVC) that is at least about 55% of the predicted normal value and/or has a carbon monoxide diffusing capacity (DL_{CO}) that is at least about 30% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration of the amount of IFN-γ to the patient in order to effect the clinical outcome of a reduction in the risk of death of the patient, where the patient has a forced vital capacity (FVC) that is at least about 55% of the predicted normal value and/or has a carbon monoxide diffusing capacity (DL_{CO}) that is at least about 35% of the predicted normal value.

In another aspect, the invention features a kit or article of manufacture comprising: (a) a container comprising an amount of IFN-γ for the treatment of a patient suffering from idiopathic pulmonary fibrosis (IPF); and (b) a label comprising printed instructions for the administration of the amount of IFN-γ to the patient in order to effect the clinical outcome of a reduction in the risk of death of the patient, where the patient has a forced vital capacity (FVC) that is at least about 55% of the predicted normal value and/or has a carbon monoxide diffusing capacity (DL_{CO}) that is at least about 30% of the predicted normal value.

In another aspect, the invention features any of the above-described kits and articles of manufacture, in which the label further comprises instructions for the administration of the amount of IFN-γ to the patient for a period of about 48 weeks. In yet other embodiments, the label comprises instructions for the administration of the amount of IFN-γ for a period of about 60 weeks. In other embodiments, the label comprises instructions for the administration of the amount of IFN-γ for a period of about one year. In still other embodiments, the label comprises instructions for the administration of the amount of IFN-γ for a period of at least about 70 weeks. In additional embodiments, the label comprises instructions for the administration of the amount of IFN-γ for a period of at least about 93 weeks. In further embodiments, the label comprises instructions for the administration of the amount of IFN-γ for a period of at least about 2 years. In still further embodiments, the label comprises instructions for the administration of the amount of IFN-γ for the remainder of the patient's life.

### BRIEF DESCRIPTION OF THE DRAWINGS

FIG. 1 depicts progression-free survival in the study patient population.

FIG. 2 depicts overall survival in the study patient population.

FIG. 3 depicts survival in the ITT population and subgroups in patients treated with IFN-γ versus placebo for 48 weeks. Patients treated with IFN-γ and having a baseline FVC of ≧55% of the predicted normal value, and a DL_{CO} of ≧35% of the predicted normal value, showed a 100% reduction in the risk of death (p=0.003).

FIG. 4 depicts survival in ITT population and subgroups in patients treated with IFN-γ versus placebo for 70 weeks. Patients treated with IFN-γ and having a baseline FVC of ≧55% of the predicted normal value, and a DL_{CO} of ≧35% of the predicted normal value, showed a 74% reduction in the risk of death (p=0.016).

FIG. 5 depicts survival in ITT population and subgroups in patients treated with IFN-γ for 93 weeks, versus placebo for 70 weeks followed by IFN-γ for 23 weeks. Patients treated with IFN-γ for 93 weeks and having a baseline FVC of ≧55% of the predicted normal value, and a DL_{CO} of ≧35% of the predicted normal value, showed a 78% reduction in the risk of death (p=0.016).

### Definitions

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, particularly a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a method” includes a plurality of such methods and reference to “an IFN-γ dose” includes reference to one or more doses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ to an individual with IPF.

METHODS OF TREATING IDIOPATHIC PULMONARY FIBROSIS

The present invention provides methods of treating idiopathic pulmonary fibrosis (IPF). The methods generally involve administering an effective amount of IFN-γ to an individual having IPF.

In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy. (or, if present, is not extensive enough to be visible on chest x-ray). A diagnosis of definite IPF is made if characteristics (1), (2), and (3) are met A diagnosis of probable EPF is made if characteristics (1) and (3) are met.

IFN-γ is administered in an effective amount. In some embodiments, an effective amount of IFN-γ is an amount effective to increase the probability of survival of an individual having IPF by at least about 10%, at least about 15%, at least about 20%, or at least about 25%, or more, compared to the expected probability of survival without administration of IFN-γ. Thus, the increased probability of survival of an individual having IPF and administered with an effective amount of IFN-γ is at least about 10%, at least about 15%, at least about 20%, or at least about 25%, or more, compared to the expected probability of survival without administration of IFN-γ.

In some embodiments, an effective amount of IFN-γ is an amount that reduces the risk of death in an individual with IPF. The risk of death in an individual having IPF and treated with IFN-γ is reduced at least 2-fold, at least 2.5-fold, at least 3-fold, at least 3.5-fold, or at least 4-fold, or less, compared to the expected risk of death in an individual having IPF and not treated with IFN-γ. In some embodiments, the risk of death in an individual having IPF and treated with IFN-γ is reduced by from about 70% to about 100%, e.g., by at least about 70% to at least about 75%, from at least about 75% to at least about 80%, from at least about 80% to at least about 90%, or from at least about 90% to about 100%.

In particular embodiments, the invention provides methods of treating IPF in an individual having IPF, where the individual has a DL_{CO} value ≧35% of predicted and has at least 55% of the predicted FVC. In particular embodiments, the invention provides a method for increasing probability of survival of an individual having IPF, where the individual has a DL_{CO} value ≧35% of predicted and has at least 55% of the predicted FVC. In these embodiments, the probability of survival is increased by at least about 10%, at least about 15%, at least about 20%, at least about 25%, or more, compared to an untreated individual. In these embodiments, the mortality is reduced by from about 70% to about 100%, e.g., by at least about 70% to at least about 75%, from at least about 75% to at least about 80%, from at least about 80% to at least about 90%, or from at least about 90% to about 100%.

In particular embodiments, the invention provides methods of treating IPF in an individual having IPF, where the individual has a DL_{CO} value ≧30% of predicted. In particular embodiments, the invention provides a method for increasing probability of survival of an individual having IPF, where the individual has a DL_{CO} value ≧30% of predicted. In these embodiments, the probability of survival is increased by at least about 10%, at least about 15%, at least about 20%, at least about 25%, or more, compared to an untreated individual. In these embodiments, the mortality is reduced by from about 70% to about 100%, e.g., by at least about 70% to at least about 75%, from at least about 75% to at least about 80%, from at least about 80% to at least about 90%, or from at least about 90% to about 100%.

In particular embodiments, the invention provides methods of treating IPF in an individual having IPF, where the individual has a DL_{CO} value ≧30% of predicted and has at least 55% of the predicted FVC. In particular embodiments, the invention provides a method for increasing probability of survival of an individual having IPF, where the individual has a DL_{CO} value ≧30% of predicted and has at least 55% of the predicted FVC. In these embodiments, the probability of survival is increased by at least about 10%, at least about 15%, at least about 20%, at least about 25%, or more, compared to an untreated individual. In these embodiments, the mortality is reduced by from about 70% to about 100%, e.g., by at least about 70% to at least about 75%, from at least about 75% to at least about 80%, from at least about 80% to at least about 90%, or from at least about 90% to about 100%.

In some embodiments, an effective amount of IFN-γ is administered to a patient having IPF for a period of time of from about 40 weeks to about 100 weeks, e.g., from about 40 weeks to about 50 weeks (e.g., for about 48 weeks), from about 50 weeks to about 55 weeks (e.g., 52 weeks), from about 55 weeks to about 60 weeks, from about 60 weeks to about 65 weeks, from about 65 weeks to about 70 weeks, from about 70 weeks to about 75 weeks, from about 75 weeks to about 80 weeks, from about 80 weeks to about 85 weeks, from about 85 weeks to about 90 weeks, from about 90 weeks to about 95 weeks, or from about 95 weeks to about 100 weeks or longer.

In some embodiments, an effective amount of IFN-γ is administered to a patient having IPF, where the individual has a DL_{CO} value ≧35% of predicted and has at least 55% of the predicted FVC, for a period of time of from about 40 weeks to about 100 weeks, e.g., from about 40 weeks to about 50 weeks (e.g., for about 48 weeks), from about 50 weeks to about 55 weeks (e.g., 52 weelks), from about 55 weeks to about 60 weeks, from about 60 weeks to about 65 weeks, from about 65 weeks to about 70 weeks, from about 70 weeks to about 75 weeks, from about 75 weeks to about 80 weeks, from about 80 weeks to about 85 weeks, from about 85 weeks to about 90 weeks, from about 90 weeks to about 95 weeks, or from about 95 weeks to about 100 weeks or longer.

In some embodiments, an effective amount of IFN-γ is administered to a patient having IPF, where the individual has a DL_{CO} value ≧30% of predicted, for a period of time of from about 40 weeks to about 100 weeks, e.g., from about 40 weeks to about 50 weeks (e.g., for about 48 weeks), from about 50 weeks to about 55 weeks (e.g., 52 weeks), from about 55 weeks to about 60 weeks, from about 60 weeks to about 65 weeks, from about 65 weeks to about 70 weeks, from about 70 weeks to about 75 weeks, from about 75 weeks to about 80 weeks, from about 80 weeks to about 85 weeks, from about 85 weeks to about 90 weeks, from about 90 weeks to about 95 weeks, or from about 95 weeks to about 100 weeks or longer.

In some embodiments, an effective amount of IFN-,Y is administered to a patient having IPF, where the individual has a DL_{CO} value ≧30% of predicted and has at least 55% of the predicted FVC, for a period of time of from about 40 weeks to about 100 weeks, e.g., from about 40 weeks to about 50 weeks (e.g., for about 48 weeks), from about 50 weeks to about 55 weeks (e.g., 52 weeks), from about 55 weeks to about 60 weeks, from about 60 weeks to about 65 weeks, from about 65 weeks to about 70 weeks, from about 70 weeks to about 75 weeks, from about 75 weeks to about 80 weeks, from about 80 weeks to about 85 weeks, from about 85 weeks to about 90 weeks, from about 90 weeks to about 95 weeks, or from about 95 weeks to about 100 weeks or longer.

The present invention provides methods of reducing the length of hospital stay in an individual having IPF and admitted to a hospital due to an IPF-related disease event. In some embodiments, the IPF-related disease event is a respiratory event. The length of hospital stay is reduced by at least about 1 day to about 25 days, e.g., by from about 1 day to about 2 days, from about 2 days to about 4 days, from about 4 days to about 6 days, from about 6 days to about 8 days, from about 8 days to about 10 days, from about 10 days to about 12 days, from about 12 days to about 15 days, from about 15 days to about 20 days, or from about 20 days to about 25 days, compared to the duration of hospital stays without IFN-γ treatment. Thus, a subject treatment method reduces the length of a hospital stay following admission to a hospital due to an IPF-related disease event by from 5% to about 80% or more, e.g., by from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 60%, from about 60% to about 70%, or from about 790% to about 80%, or more, compared to the length of hospital stay in a patient not treated with IFN-γ.

KITS

The invention fttther provides a kit comprising a formulation comprising a unit dosage form of IFN-γ in a container, and a label that provides instructions for use of the kit. The container comprises a formulation comprising IFN-γ in a unit dosage form of from about 25 μg to about 500 μg, from about 50 μg to about 400 μg, or from about 100 μg to about 300 μg. In particular embodiments of interest, the dose is about 100 μg IFNγ. In other particular embodiments of interest, the dose is about 200 μg IFN-γ. In many embodiments, the IFN-γ is IFN-γ2b.

Suitable containers include those adapted for administration by subcutaneous injection, including a syringe (for use with a needle), an injector pen, and the like. In some embodiments, IFN-γ is administered with a pen injector (e.g., a medication delivery pen), a number of which are known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The label provides written instructions for use of the kit. The label includes instructions for the administration of the amount of IFN-γ to the patient for a period of about 48 weeks. In some embodiments, the label comprises instructions for the administration of the amount of IFN-γ for a period of about 60 weeks. In other embodiments, the label comprises instructions for the administration of the amount of IFN-γ for a period of about one year. In still other embodiments, the label comprises instructions for the administration of the amount of IFN-γ for a period of about 70 weeks. In additional embodiments, the label comprises instructions for the administration of the amount of IFN-γ for a period of about 93 weeks. In further embodiments, the label comprises instructions for the administration of the amount of IFN-γ for a period of at least about 2 years. In still further embodiments, the label comprises instructions for the administration of the amount of IFN-γ for the remainder of the patient's life.

INTERFERON-GAMMA

The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J. Biol. Chem. 259:6790.

IFN-γ1b (Actimmune®; human interferon) is a single-chain polypeptide of 140 amino acids. It is made recombinantly in E.coli and is unglycosylated. Rinderknecht et al. (1984) J. Biol. Chem. 259:6790-6797.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression.

The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151,24-152; 24- 153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001 or WO 02/081507. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

DOSAGES, FORMULATIONS, AND ROUTES OF ADMINISTRATION

IFN-γ is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal,etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle.

The specifications for the novel unit dosage forms of the present invention depend on the particular compound employed and the effect to be achieved, and the pharmacodynamics associated with each compound in the host.

Effective dosages of IFN-γ can range from about 0.5 μg/m² to about 500 μg/m², usually from about 1.5 μg/m² to 200 μg/m², depending on the size of the patient. This activity is based on 10⁶ international units (IU) per 50 μg of protein.

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means is to measure the physiological potency of a given compound.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individias generally ranges from about 1.33 m² to about 2.50 m². Thus, dosage groups (based on administration of 200 μg IFN-γ per dose) range from about 150 μg/m² to about 80 μg/m². For example, dosage groups range from about 80 μm² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μ/m² to about 150 μg/m².

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide (e.g., a polynucleotide encoding IFN-γ), it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold ricroparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells. Of particular interest in these embodiments is use of a liver-specific promoter to drive transcription of an operably linked IFN-γ coding sequence preferentially in liver cells.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

In particular embodiments of interest, IFN-γ is administered as a solution suitable for subcutaneous injection. For example, IFN-γ is in a formulation containing 40 mg mannitol/mL, 0.72 mg sodium succinate/mL, 0.10 mg polysorbate 20/mL. In particular embodiments of interest, IFN-γ is administered in single-dose forms of 200 μg/dose subcutaneously.

Multiple doses of IFN- can be administered, e.g., IFN-γ can be administered once per month, twice per month, three times per month, once per week, twice per week, three times per week, four times per week, five times per week, six times per week, or daily, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In particular embodiments of interest, IFN-γ is administered three times per week over a period of at least about 1 year.

In some embodiments, IFN-γ is co-administered with one or more additional agents. Suitable additional agents include corticosteroids, such as prednisone. When co-administered with IFN-γ, prednisone is administered in an amount of 7.5 mg or 15 mg daily, administered orally.

SUBJECTS SUITABLE FOR TREATMENT

The subject methods are suitable for treatment of individuals diagnosed as having IPF. The methods are also suitable for treatment of individuals having IPF who were previously treated with corticosteroids within the previous 24 months, and who failed to respond to previous treatment with corticosteroids. Subjects that are particularly amenable to treatment with a method are those that have at least 55% of the predicted FVC. Also suitable for treatment are subject that have at least 60% of the predicted FVC, or from 55% to 70% of the predicted FVC. The percent predicted FVC values are based on normal values, which are known in the art. See, e.g., Crapo et al. (1981) Anm. Rev. Respir. Dis. 123:659-664. FVC is measured using standard methods of spirometry.

Other subjects that are suitable for treatment have carbon monoxide diffusing capacity (DL_{CO}) ≧25% of predicted, e.g., ≧35% of predicted.

Other subjects that are suitable for treatment have DL_{CO} ≧35% of predicted and at least 55% of the predicted FVC.

Other subjects that are suitable for treatment have carbon monoxide diff-using capacity (DL_{CO}) ≧30% of predicted. Other subjects that are suitable for treatment have DL_{CO} ≧30% of predicted and at least 55% of the predicted FVC.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Controlled Trial of IFN-γ1b for Idiopathic Pulmonary Fibrosis

PATIENTS AND METHODS

Patient Population

Between September 2000 and October 2001, 330 patients from 58 centers in the United States, Europe, Canada and South Africa were randomized into the study. The diagnosis of IPF was established according to previously described clinical, radiologic and histologic criteria⁴ Eligible patients were those aged 20-79 years with clinical symptoms of IPF for 2 3 months, forced vital capacity (FVC) 50-90% of predicted, carbon monoxide diffusing capacity (DL_{CO}) ≧25% of predicted, and room air pO₂>55 mmHg at rest (or pO₂>50 mmHg if altitude >4000 feet). The diagnosis of IPF was confirmed by high-resolution computerized tomography (HRCT) scan showing definite or probable IPF by pre-specified criteria, plus either surgical biopsy (open-lung or video-assisted thoracoscopic) showing usual interstitial pneumonia (UIP) or transbronchial biopsy to exclude other conditions. Other entry requirements included worsening of IPF within the preceding year (210% decrease in % predicted FVC, worsening chest X-ray, or worsening dyspnea) plus lack of improvement (<10% increase in % predicted FVC) during receipt of ≧1800 mg total of prednisone or equivalent within the 24 months prior to entry. Patients taking prednisone (≦15 mg/d) were eligible for entry into the study provided that the dose could be held constant throughout the study. All patients provided written informed consent at enrollment and the Institutional Review Board at each center approved the protocol.

Patients with any of the following were excluded: significant exposure to known fibrogenic agents, alternative etiology for interstitial lung disease, FEV₁ (forced expiratory volume in 1 second)/FVC <0.6 after bronchodilator administration, residual volume >120% predicted, active infection within 1 week preceding entry, unstable cardiovascular or neurologic disease, uncontrolled diabetes, pregnancy, lactation, or the likelihood of death within the next year. Laboratory results mandating exclusion were total bilirubin ≧1.5×upper limit normal (ULN); aspartate transaminase or alanine transaminase >3×ULN; alkaline phosphatase >3×ULN; creatinine >1.5×ULN, albumin <3.0 mg/dL, white blood cell count <2,500×10⁹/L, hematocrit <30% or >59%, and platelet count <100,000×10⁹/L. Prior treatment with any interferon was prohibited, as was use of azathioprine, coichicine, cyclophosphamide, cyclosporine, D-penicillamine, methotrexate, or N-acetyl cysteine within 6 weeks of treatment, investigational therapy for IPF within 28 days of entry, and other investigational therapies within the preceding 6 months.

Study Design

Patients were randomized 1:1 to receive IFN-γ1b or matching placebo, administered subcutaneously three times weekly. The randomization was stratified by cigarette smoking status and blocked by study center.

The dose of study drug was increased from 100 μg to 200 μg after 2 weeks. Bedtime administration of the study drug was recommended and pre-treatment with acetaminophen or ibuprofen required. Patient compliance was actively monitored through review of recorded injections in patient diaries and by the counting of all used medication vials. Oxygen use was also recorded in patient diaries.

Hematologic and serum chemistry tests were collected serially. After baseline measurements, arterial blood gases at rest, pulmonary function tests (FVC, FEV₁, DL_{CO} corrected to hematocrit), St. George's Respiratory Questionnaire (SGRQ),¹⁹ and the Baseline Dyspnea Index/Transition Dyspnea Index (BDI/TDI)²⁰ were performed at 3-month intervals; measurement of TLC by body box plethysmography, chest x-ray, and HRCT scan were repeated at Week 48. Two expert chest radiologists who were blinded to patient identification, treatment assignment, and temporal sequence of the studies performed central and independent scoring of the extent of lung fibrosis (including honeycombing and reticular abnormalities) on the two HRCT images from each patient. A third radiologist scored in the event of discrepant interpretation.

Statistical Issues

The primary efficacy endpoint was progression-free survival time. Progression was defined as either of the following changes from baseline, confirmed on the next visit 4-14 weeks later: ≧10% decrease in % predicted FVC or 25 mmHg increase in arterial-alveolar (A-a) gradient. Progression-free survival time was measured from randomization and compared between treatment groups using the likelihood ratio test from the Cox proportional hazards model, with treatment group and baseline smoking status as covariates.

Survival time was compared between treatment groups as a secondary endpoint, using the log-rank test stratified by smoking status in two pre-specified analyses: (1) all randomized patients; (2) the compliant patient cohort (i.e., patients receiving ≧80% of scheduled study drug doses). Exploratory subgroup analyses of survival were based on baseline measures of disease severity (e.g., % predicted FVC and DL_{CO}) and divided the population at the median values. Multivariate analysis of covariates used Cox proportional hazards regression.

Other secondary endpoints compared the change from baseline to Week 48 in dyspnea (i.e., the TDI), DL_{CO}, FVC, A-a gradient, SGRQ total score, and lung fibrosis on HRCT (better, same, worse). Two other secondary endpoints were the most severe monthly requirement for supplemental oxygen use (i.e., none, with activity, at rest) and analysis of progression-free survival using an alternate definition of disease progression (any two of the following: ≧10% decrease in % predicted FVC, ≧5 mmHg increase in A-a gradient, or ≧15% decrease in DL_{CO}).

Final analysis of the data occurred at a pre-specified timepoint: 48 weeks after the 306^{th} patient was randomized. Efficacy analyses included all patients who were randomized with intention to treat. Safety analyses included all patients receiving at least one dose of study drug. Adverse events were graded according to the modified Common Toxicity Criteria of the National Institutes of Health and coded according to MedDRA preferred terms. Analysis of continuous variables used the analysis of covariance, with effects for treatment, age, sex, height, baseline value, and the inverse of baseline hemoglobin (for DL_{CO}) or race (for FVC); for change in A-a gradient only treatment and baseline value were included in the model. Categorical variables were analyzed using the Cochran-Mantel-Haenszel row mean scores test, stratified by smoking status. Final (i.e., “endpoint”) evaluations were used to incorporate data from dropouts, with values carried forward from the date of last visit.

The planned sample size of 306 patients was selected to provide 94% power to detect a difference in progression-free survival time equivalent to a 20% reduction in the rate of death or disease progression at 1 year (i.e. 40% to 20%), using a two-tailed test at the 5% significance level. An independent Data Monitoring Committee regularly reviewed emerging safety and efficacy data. Patients were to continue on blinded therapy for ˜3- 4 months after primary analysis of the study. Mortality is to be monitored for a total of 5 years from the date of randomization in all patients.

RESULTS

Of the 330 patients randomized into the study, 162 received IFN-γ 1b and 168 received placebo. No imbalances in clinically relevant baseline characteristics were apparent (Table 1). The majority of patients were non-smolkers (93%), Caucasian (89%), male (68%), and aged 61 to 80 years (66%). The median time since diagnosis of IPF was 312 days. Most patients were taking prednisone (76%) but did not use supplemental oxygen (58%). Baseline lung function was similar in both groups, demonstrating reduced lung volumes and abnormal gas exchange. The diagnosis of IPF was confirmed by identification of UIP on surgical lung biopsy in 62% IFN-γ lb and 67% placebo patients, respectively. HRCT scans were interpreted as definite IPF (see Methods) in 84% vs. 83% of patients, respectively.

TABLE 1
Characteristics of the Study Population at Entry

| | IFN-γ 1b | Placebo | |
|---|---|---|---|
| Characteristic | (N = 162) | (N = 168) | P value^{a} |
| Age (years)^{b} | 63.6 ± 8.6 | 63.4 ± 8.6 | 0.8 |
| Proportion of men (%) | 71.6 | 65.5 | 0.2 |
| Ethnicity (%) | | | 0.1 |
| Caucasian | 91.4 | 86.3 | |
| Black | 1.9 | 5.4 | |
| Asian | 0 | 3.0 | |
| Hispanic | 4.9 | 4.2 | |
| Other | 1.9 | 1.2 | |
| Smoking status^{c} (%) | | | 0.2 |
| Non-smokers | 95.1 | 91.1 | |
| Smokers | 4.9 | 8.9 | |
| Days since IPF diagnosis^{b} | 425.3 ± 368.6 | 378.2 ± 295.2 | 0.2 |
| Arterial pO₂ at rest (mmHg)^{b} | 73.5 ± 10.2 | 74.1 ± 10.3 | 0.6 |
| FVC (% of predicted)^{b} | 63.9 ± 10.7 | 64.1 ± 11.3 | 0.9 |
| DL_{co} (% of predicted)^{b} | 37.2 ± 11.2 | 36.8 ± 10.6 | 0.7 |
| Use of prednisone or | 75.3 | 77.4 | 0.9 |
| equivalent (%) | | | |
| Use of supplemental oxygen | 40.7 | 31.0 | 0.1 |
| (%) | | | |

^{a}p-value is based on t-tests for continuous data and chi-square tests for categorical data
^{b}mean ± standard deviation
^{c}smokers were defined as those currently smoking or those who smoked within the year prior to study entry

The median treatment durations were 383 (range, 13-643) and 374 (range, 12646) days in EFN-γ1b and placebo patients, respectively. Adherence was high: an average of 93% of all scheduled doses were received, and 90% of patients complied with protocol follow-up visits through study end, even if discontinuing treatment. Sixty (33 IFN-γ1b, 27 placebo) of the 330 (18%) randomized patients discontinued study drug treatment prematurely, due to: patient request for withdrawal: 16 vs. 16 patients, respectively; adverse event, 8 vs. 2; lung transplantation, 5 vs. 1; other reason, 1 vs. 4; investigator discretion, 3 vs. 3; use of prohibited therapy, 0 vs. 1. Seven patients (1 IFN-γ1b, 6 placebo) who discontinued blinded study drug initiated therapy with open-label IFN-γ1b.

Disease Progression and Mortality

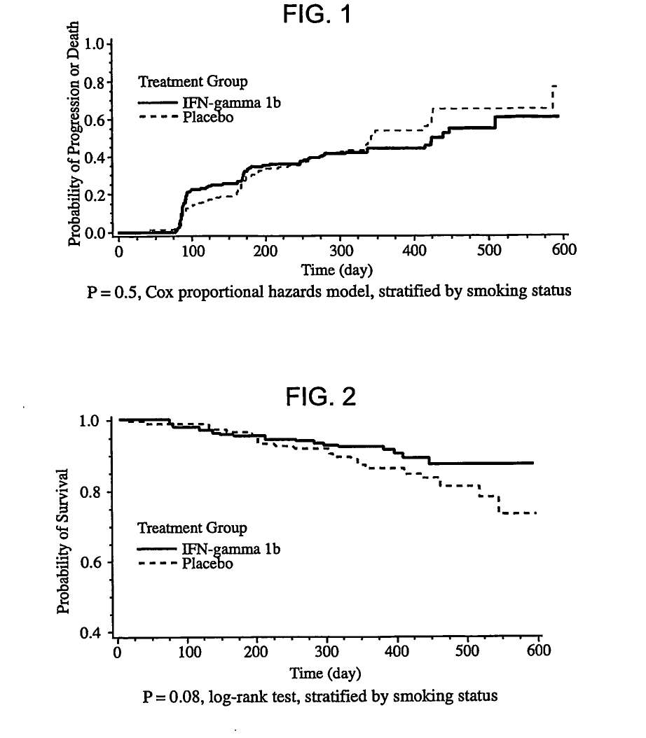

In the primary efficacy analysis, there was no significant difference in progression-free survival time in the IFN-γ1b and placebo groups (median time to death or disease progression, 439 and 344 days, respectively; P=0.5, Cox proportional hazards model; FIG. 1). Death or disease progression occurred in 46.3% vs. 51.8% of IFN-γ1b and placebo patients, respectively (Table 2). The majority of primary endpoint events were disease progression rather than death (88%), and the majority of disease progression events in both treatment groups (62%) were increases in A-a gradient.

TABLE 2
Progression-free Survival

| | IFN-γ 1b | Placebo | |
|---|---|---|---|
| | (N = 162) | (N = 168) | P value^{a} |
| Death or Disease | 75 (46.3%) | 87 (51.8%) | 0.5 |
| Progression^{b} | | | |
| Disease progression | 68 (42.0%) | 75 (44.6%) | |
| Increase in A-a gradient | 43 (26.5%) | 46 (27.4%) | |
| Decrease in FVC | 8 (4.9%) | 12 (7.1%) | |
| Both | 17 (10.5%) | 17 (10.1%) | |
| Death without disease | 7 (4.3%) | 12 (7.1%) | |
| progression | | | |

^{a}p value is derived from the likelihood ratio test from the Cox proportional hazards model, stratified by smoking status
^{b}the occurrence of death or disease progression was the primary endpoint of the study. Disease progression was defined as either of the following occurrences on two consecutive occasions 4-14 weeks apart compared to baseline: ≧10% decrease in % predicted FVC or ≧5 mmHg increase in A-a gradient.

Vital status was ascertained in all enrolled patients for mortality analysis. Sixteen of 162 (9.9%) of IFN-γ lb patients and 28 of 168 (16.7%) placebo patients died, representing a 41% relative reduction in the risk of death (P=0.08, stratified log-rank test; FIG. 2). A pre-specified analysis of the compliant patient cohort (i.e. including only those patients who received ≧80% of scheduled study drug doses) found a stronger treatment effect on survival, with a 72% reduction in the risk of death: 5 (4%) of 125 IFN-γ1b vs. 19 (13.4%) of 142 placebo patients, respectively (P=0.01, stratified log-rank test). Exploratory subgroup analyses that dichotomized baseline lung function by median values suggested that patients with less severe lung function impairment had a greater impact of treatment on survival. In patients with baseline FVC 262% predicted, death occurred in 3.5% of 86 IFN-γ1b vs. 12.5% of 88 placebo patients (P=0.04). Conversely, in patients with baseline FVC <62% (n=156), survival benefit was not apparent (death in 17.1% vs. 21.3%, respectively; P=0.6). In patients entering the study with DL_{CO}≧35% of predicted, mortality rates were 4.6% vs. 12.9%, respectively; P=0.06); in those with baseline DL_{CO}<35%, mortality rates were 16.0% vs. 20.5%; P=0.5. In a multivariate analysis, compliance with study drug and baseline FVC were shown to be independent predictors of survival, as was study drug treatment.

The reported cause of death was related to the respiratory tract in ˜80% of patients in each treatment group. Of these, respiratory insufficiency comprised 38% and 39%, respectively, of respiratory deaths in the IFN-γ1b and placebo groups, and progression of IPF comprised 38% and 48%, respectively. Duration of disease, gender, definite diagnosis of IPF on HRCT, mode of histopathologic diagnosis of IPF, and use of prednisone during the study period did not affect treatment group differences in survival.

No treatment effect was discernable in the mean change between baseline and Week 48 in FVC, DL_{CO}, A-a gradient, change in lung fibrosis on HRCT, or using a pre-specified alternate definition of progression-free survival.

Dyspnea and Quality of Life

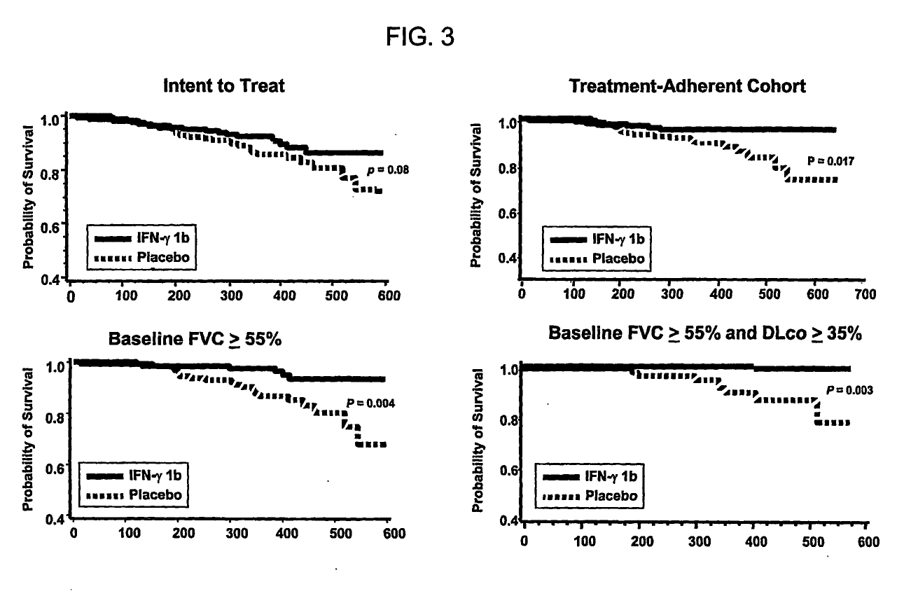

Dyspnea, as assessed by either the TDI at Week 48 or mean change from baseline to Week 48 in SGRQ total score, showed no significant treatment effect. However, divergence in TDI scores of the two treatment groups appeared to begin at Week 48 and widen thereafter, although the numbers of patients at each timepoint after Week 48 were small (FIG. 3).

Although use of supplemental oxygen was somewhat more frequent at baseline in patients receiving IFN-γ 1b (41% vs. 31%; Table 1), fewer IFN-γ 1b patients initiated new use of oxygen during the study than did placebo patients (21% vs. 35%; P=0.1).

Table 3 provides the data for survival in ITT population and subgroups. Table 3 depicts survival in the ITT population and subgroups in patients treated with IFN-γ versus placebo for 48 weeks. Patients treated with IFN-γ and having a baseline FVC of ≧55% of the predicted normal value, and a DL_{CO} of ≧35% of the predicted normal value, showed a 100% reduction in the risk of death (p=0.003).

TABLE 3

| | IFN-γ 1b | | Placebo | | | |
|---|---|---|---|---|---|---|
| Patient | | Deaths | | Deaths | % Reduction | p- |
| Cohort | N | (%) | N | (%) | in Mortality | value |
| ITT | 162 | 16 (9.9%) | 168 | 28 | 41% | 0.08 |
| | | | | (16.7%) | | |
| Adherent | 126 | 6 (4.8%) | 143 | 20 | 66% | 0.017 |
| | | | | (14.0%) | | |
| FVC ≧55% | 126 | 6 (4.8%) | 128 | 21 | 71% | 0.004 |
| | | | | (16.4%) | | |
| DLco ≧35% | 87 | 4 (4.6%) | 85 | 11 | 64% | 0.057 |
| | | | | (12.9%) | | |
| FVC ≧55% | 71 | 0 (0.0%) | 68 | 8 | NA | 0.003 |
| and | | | | (11.8%) | | |
| DLco ≧35% | | | | | | |

Safety

The incidence of treatment-emergent adverse events was high: 99% vs. 98% in IFN-γ 1b vs. placebo patients, respectively (Table 4). The most common adverse events in both groups were headache, cough and upper respiratory tract infection. Constitutional symptoms such as fever, rigor, influenza-like illness, back pain, arthralgias and myalgias were more common in IFN-γ 1b patients. Nausea and/or vomiting and dizziess were more frequent in placebo patients. Adverse events graded as severe or life-threatening events occurred in 44% vs. 34% of IFN-γ 1b and placebo patients, respectively. Those occurring in ≧5% of patients in either treatment group were: hyperglycemia (serum glucose ≧13.9 mmol/L; 8.6% IFN-γ 1b vs. 6.0% placebo), pneumonia (6.2% vs. 4.8%), and lymphopenia (absolute lymphocyte count <500×10⁹/L; 6.2% vs. 2.4%).

TABLE 4
Treatment-emergent Adverse Events Occurring in ≧15% of Patients

| | IFN-γ 1b | | Placebo | |
|---|---|---|---|---|
| | (N = 162) | | (N = 168) | |
| Number (%) of patients with any | 161 | (99.4) | 165 | (98.2) |
| treatment-emergent adverse event | | | | |
| Headache¹ | 86 | (53.1) | 52 | (31.0) |
| Upper respiratory tract | 82 | (50.6) | 63 | (37.5) |
| infection² | | | | |
| Cough³ | 59 | (36.4) | 59 | (35.1) |
| Fever | 53 | (32.7) | 16 | (9.5) |
| Rigors | 53 | (32.7) | 15 | (8.9) |
| Fatigue⁴ | 39 | (24.1) | 33 | (19.6) |
| Dyspnea⁵ | 39 | (24.1) | 43 | (25.6) |
| Pain | 37 | (22.8) | 23 | (13.7) |
| Diarrhea⁶ | 37 | (22.8) | 35 | (20.8) |
| Arthralgia | 33 | (20.4) | 23 | (13.7) |
| Influenza-like illness | 31 | (19.1) | 13 | (7.7) |
| Myalgia | 30 | (18.5) | 15 | (8.9) |
| Nausea and/or vomiting⁷ | 29 | (17.9) | 49 | (29.2) |
| Back pain | 29 | (17.9) | 20 | (11.9) |
| Chest pain | 26 | (16.0) | 27 | (16.1) |
| Nasal congestion | 25 | (15.4) | 26 | (15.5) |
| Bronchitis⁸ | 25 | (15.4) | 29 | (17.3) |
| Dizziness | 18 | (11.1) | 29 | (17.3) |

¹includes headache, aggravated headache, migraine, and sinus headache
²includes upper respiratory tract infection, viral upper respiratory tract infection, sinusitis, acute sinusitis, otitis media, ear infection, laryngitis, nasopharyngitis, streptococcal pharyngitis
³includes cough, aggravated cough, and productive cough
⁴includes fatigue and aggravated fatigue
⁵includes dyspnea, exacerbated dyspnea, and exertional dyspnea
⁶includes diarrhea and aggravated diarrhea
⁷includes nausea, aggravated nausea, and vomiting
⁸includes bronchitis, acute bronchitis, acute exacerbation of chronic bronchitis, and tracheobronchitis

Respiratory tract infections were frequent, occurring in 67.9% of IFN-γ 1b patients and in 56.5% of placebo patients overall. Of these, pneumonias comprised 14.8% vs. 8.3%, respectively, and unspecified respiratory tract infections, 11.7% vs. 11.3%. Respiratory tract infections that were graded by the investigator as severe or life-threatening were reported in 13 (8.0%) IFN-γ 1b and 14 (8.3%) placebo patients. Twenty-nine respiratory tract infections resulted in hospitalization in 26 (16.0%) IFN-γ 1b patients, as did 19 events in 16 (9.5%) placebo patients. Respiratory tract infections that resulted in death occurred in 3 patients in each treatment group. Only one respiratory infection, an episode of acute bronchitis/pneumonia in a patient receiving placebo, resulted in withdrawal from study drug treatment.

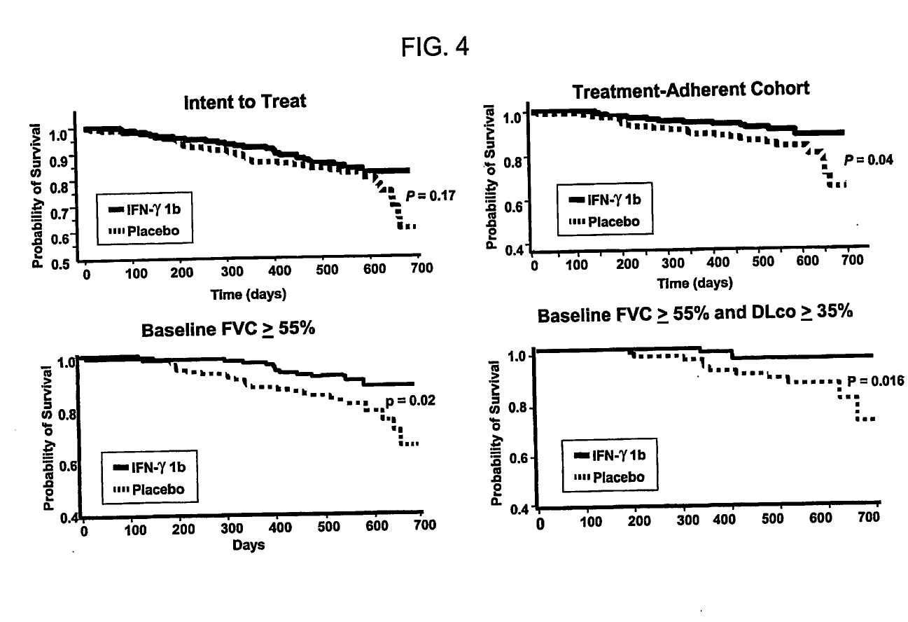

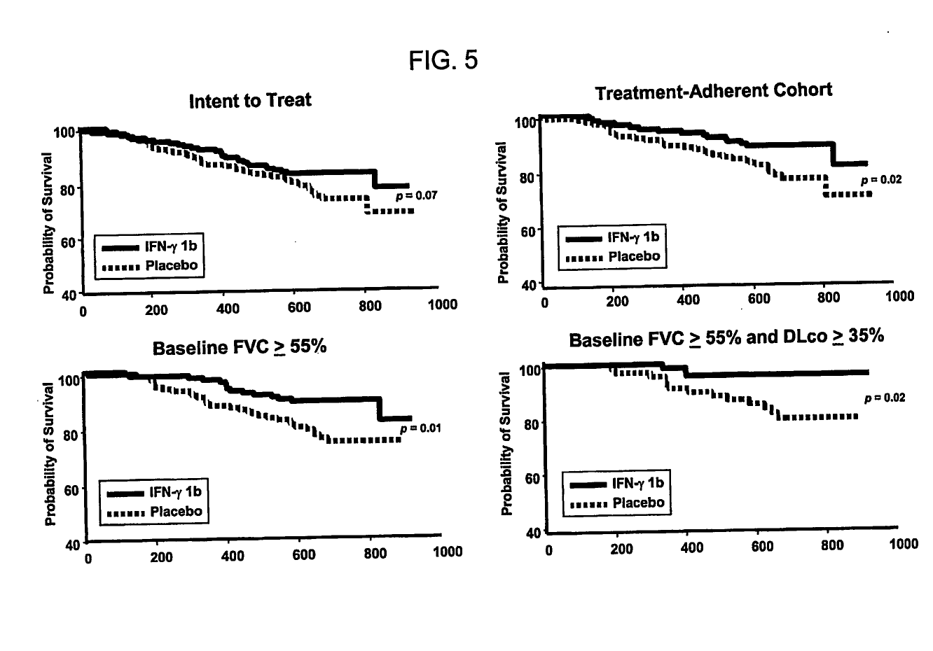

Additional data are shown in FIG. 4 and Table 5; and FIG. 5 and Table 6.

Table 5 depicts duration of hospitalizations with respiratory admission diagnosis in ITT population and a subgroup treated with IFN-γ versus placebo for 70 weeks. Patients treated with IFN-γ and having a baseline FVC of ≧55% of the predicted normal value showed a 43% reduction in mean duration of hospitalizations with respiratory admissions (p=0.038).

TABLE 5

| | IFN-γ 1b | Placebo | |
|---|---|---|---|
| | (n = 162) | (n = 168) | p-value |
| Number of Admissions (ITT) | 59 | 57 | |
| Duration (days) | | | |
| Mean | 11.4 | 15.0 | 0.100 |
| Median | 8.0 | 10.5 | |
| Number of Admissions (FVC ≧55%) | 34 | 36 | |
| Duration (days) | | | |
| Mean | 9.2 | 16.3 | 0.038 |
| Median | 7.0 | 10.5 | |

Table 6 depicts survival in ITM population and subgroups in patients treated with IFN-γ for 93 weeks, versus placebo for 70 weeks followed by IFN-γ for 23 weeks. Patients treated with IFN-γ for 93 weeks and having a baseline FVC of ≧55% of the predicted normal value, and a DL_{CO} of ≧35% of the predicted normal value, showed a 78% reduction in the risk of death (p=0.016).

TABLE 6

| | IFN-γ 1b | | Placebo | | | |
|---|---|---|---|---|---|---|
| | | Deaths | | Deaths | % Reduction | |
| Patient Cohort | N | (%) | N | (%) | in Mortality | p-value |
| ITT | 162 | 29 (17.9%) | 168 | 43 (25.6%) | 31% | 0.073 |
| Adherent (Nov 30) | 124 | 15 (12.1%) | 137 | 31 (22.6%) | 46% | 0.021 |
| FVC ≧55% | 126 | 15 (11.9%) | 128 | 31 (24.2%) | 51% | 0.009 |
| Dlco ≧35% | 87 | 8 (9.2%) | 85 | 18 (21.1%) | 56% | 0.026 |
| FVC ≧55% | 71 | 3 (4.2%) | 68 | 13 (19.1%) | 78% | 0.016 |
| and Dlco | | | | | | |
| ≧35% | | | | | | |

### Example 2

### Analyses of Efficacy Endpoints

The data from the trial described in Example 1 were re-analyzed. 28

Patients and Study Design

Example 1 describes a randomized study comparing subcutaneous IFN-γ 1b (200 μg; n=162) with placebo (n=168), administered three times weekly, in 330 patients who met the diagnostic criteria for IPF according to the American Thoracic Society. Eligible patients were aged 20-79, had mild-to-moderate IPF (e.g., FVC 50-90% of predicted and DLCO ≧25% of predicted), had definite or probable IPF on high-resolution computerized tomography (HRCT) scan based on protocol-specified criteria, and had worsening IPF during the preceding year despite therapy with corticosteroids. FVC, DLCO, and resting arterial blood gases at ambient temperature were measured at 3-month intervals. The median duration of follow-up was 58 weeks (range, 2-92 weeks).

Statistical Analysis

The primary efficacy endpoint was progression-free survival time, defined as time from baseline to the first occurrence of either death or disease progression. Disease progression was defined as either an increase of at least 5 mm Hg in P(A-a)O₂ or a decrease of at least 10% in % predicted FVC compared to that at baseline. Threshold changes in P(A-a)O₂ and % predicted FVC required confirmation at a subsequent visit within 4-14 weeks. Vital status was ascertained in all randomized patients at the completion of the trial.

A series of analyses was conducted that examined the rates of primary endpoint events in all randomized patients. First, the association of single components or combinations of components on the rate of endpoint events was examined. Next, the impact of different thresholds in the components on the rate of primary endpoint events was assessed by progressively increasing the level of change from baseline (P[A-a]O₂ in 5-mm increments, % predicted FVC in 5% increments) while holding the other measurements constant or by testing the parameter alone. These analyses assessed only placebo patients in order to avoid obfuscation by a treatment effect. Endpoint reliability (i.e., reproducibility) was assessed by examining serial measurements of P(A-a)O₂ and % predicted FVC between the screening and baseline visits (i.e., prior to therapeutic intervention). The relationships between change in P(A-a)O₂, change in % predicted FVC, and change in % predicted DLCO and death were examined. The risk of death according to different thresholds of change.in P(A-a)O₂, % predicted FVC, and % predicted DLCO was calculated as a ratio relative to the reference group (i.e., least change) in patients receiving placebo. Lastly, the sensitivity of treatment effect was evaluated by comparing disease progression and mortality rates according to treatment group in various subgroups defined by baseline physiologic parameters.

RESULTS

Components of the Primary Efficacy Endpoint

A primary endpoint event (i.e., either disease progression according to change in P(A-a)O₂ or % predicted FVC criteria, or death; see Methods) occurred in 75 (46.3%) IFN-γ 1b and 87 (51.8%) placebo patients (p=0.53, likelihood score test from the Cox proportional hazards model) (Table 7).

TABLE 7
Components of the Primary Efficacy Endpoint* †

| | IFN-γ 1b | Placebo |
|---|---|---|
| | (n = 162) | (n = 168) |
| Disease progression‡ | 68 (42.0%) | 75 (44.6%) |
| Increase in P(A-a)O₂ | 43 (26.5%) | 46 (27.4%) |
| Decrease in % predicted FVC | 8 (4.9%) | 12 (7.1%) |
| Both P(A-a)O2 increase and % | 17 (10.5%) | 17 (10.1%) |
| predicted FVC decrease | | |
| Death prior to disease | 7 (4.3%) | 12 (7.1%) |
| progression | | |
| Total | 75 (46.3) | 87 (51.8) |

*P(A-a)O₂ = alveolar-arterial oxygen pressure gradient.
† The primary efficacy endpoint, progression-free survival time, was defined as the time to first occurrence of disease progression or death during the study period.
‡Disease progression was defined as either a ≧10% decrease in % predicted FVC or a ≧5 mm Hg increase in P(A-a)O₂ compared to baseline, on two consecutive occasions 4-14 weeks apart.

The majority of study patients reaching the primary endpoint did so on the basis of disease progression rather than death (90.7% IFN-γ 1b and 86.2% placebo), and the majority of these events were due to a protocol-specified increase in P(A-a)O₂ (43 of 75 events in IFN-γ 1b patients and 46 of 87 events in placebo patients). The frequency of FVC-dependent endpoints was considerably lower in both treatment groups: 8 of 75 events in the IFN-γ 1b group and 12 of 87 in the placebo group. Concurrent changes in both P(A-a)O₂ and % predicted FVC occurred in 17 subjects in each treatment group, while death prior to documentation of disease progression occurred in 7 IFN-γ 1b and 12 placebo patients.

Exploration of the Components of the Primary Endpoint

Effects of Varying the Threshold Levels of Physiologic Parameters

When the definition of the primary endpoint included only the P(A-a)O₂ criterion and death, 78 placebo patients met the endpoint, a 10% reduction compared with the number of patients meeting the endpoint using the original protocol definition (Table 8).

TABLE 8
Comparison of Outcomes When Varying the Primary
Efficacy Endpoint Definition* †

| | | Number (%) of Patients | |
|---|---|---|---|
| | | Meeting the Endpoint | |
| | | IFN-γ 1b | Placebo |
| | Variation | (n = 162) | (n = 168) |
| Outcome according to | — | 75 (46.3%) | 87 (51.8%) |
| original protocol | | | |
| definition (decrease | | | |
| in % predicted | | | |
| FVC ≧10%, | | | |
| increase in P(A-a)O2 ≧5 | | | |
| mm Hg, or death) | | | |
| Outcomes using fewer | ↑ P(A-a)O₂ ≧5 | 68 (42.0%) | 78 (46.4%) |
| definition components | mm Hg or | | |
| | death | | |
| | ↓ % predicted | 36 (22.2%) | 48 (28.6%) |
| | FVC ≧10% or | | |
| | death | | |
| | ↑ P(A-a)O_{2 ≧5} | 68 (42.0%) | 75 (44.6%) |
| | mm Hg or ↓ | | |
| | % predicted | | |
| | FVC ≧10% | | |
| | Death alone | 16 (9.9%) | 28 (16.7%) |
| Outcomes using varying | ≧5 | 75 (46.3%) | 87 (51.8%) |
| thresholds for increase in | ≧10 | 50 (30.9%) | 65 (38.7%) |
| P(A-a)O₂ (mm Hg) while | ≧15 | 39 (24.1%) | 50 (29.8%) |
| holding other | ≧20 | 36 (22.2%) | 49 (29.2%) |
| components constant (per | ≧25 | 36 (22.2%) | 48 (28.6%) |
| original definition) | | | |
| Outcomes using varying | ≧10 | 75 (46.3%) | 87 (51.8%) |
| thresholds for decrease in | ≧15 | 70 (43.2%) | 79 (47.0%) |
| % predicted FVC (%) | ≧20 | 68 (42.0%) | 78 (46.4%) |
| while holding other | ≧25 | 68 (42.0%) | 78 (46.4%) |
| components constant (per | ≧30 | 68 (42.0%) | 78 (46.4%) |
| original definition) | | | |
| Outcomes using only | ≧5 | 60 (37.0%) | 63 (37.5%) |
| increase in P(A-a)O₂ | ≧10 | 26 (16.0%) | 29 (17.3%) |
| (mm Hg) | ≧15 | 8 (4.9%) | 12 (7.1%) |
| | ≧20 | 4 (2.5%) | 5 (3.0%) |
| | ≧25 | 0 (0.0%) | 1 (0.6%) |
| Outcomes using only | ≧10 | 25 (15.4%) | 29 (17.3%) |
| decrease in % predicted | ≧15 | 7 (4.3%) | 9 (5.4%) |
| FVC (%) | ≧20 | 1 (0.6%) | 1 (0.6%) |
| | ≧25 | 1 (0.6%) | 1 (0.6%) |

*P(A-a)O₂ = alveolar-arterial oxygen gradient.
† Confirmed on two consecutive visits 4-14 weeks apart.

In contrast, when the definition of the endpoint included only the FVC-based criterion and death, 48 placebo patients met the endpoint, a 45% reduction compared with the original endpoint definition. Thus the proportion of patients with the protocol-specified change in P(A-a)O₂ was substantially higher than those with the pre-specified change in % predicted FVC.

As the definition for disease progression was varied by progressively increasing the threshold for change in P(A-a)O₂ from baseline in 5-mm increments (while holding other measures constant), the number of endpoint events decreased substantially in the placebo group initially (87 events at the ≧5 mm Hg threshold versus 65 events at ≧10 mm Hg increase) but remained relatively constant at thresholds ≧15 mm Hg (Table 8). In contrast, incremental increases of 5% in the threshold for change in % predicted FVC resulted in only a small reduction in the number of events initially (87 events at ≧10% decrease versus 79 events at ≧1 5%) and had minimal effect at higher threshold levels (i.e., 78 events at thresholds of ≧20%, ≧25%, and ≧30% decrease).

When P(A-a)O₂ and % predicted FVC were analyzed as sole measures of the efficacy endpoint, it was found that 37.5% of placebo patients experienced an increase of ≧5 mm Hg in P(A-a)O₂ during the study, with progressively fewer patients manifesting greater changes in P(A-a)O₂ (Table 8). For example, only 7.1% of placebo patients had a ≧15 mm Hg increase in P(A-a)O₂. In contrast, 17.3% of placebo patients had a decrease of ≧10% in % predicted FVC during the study, and <1% had decreases of ≧20%.

Reliability of P(A-a)O₂ and % Predicted FVC

Changes in P(A-a)O₂ and % predicted FVC between the screening visit and the baseline visit were assessed, with a median duration of this interval of 20-21 days (range, 3 to 57 days). Since repeating these tests was not required by the protocol, only a subset of patients was available for analysis. It was found that 31 of 73 (42%) subjects tested had a change of ≧5 mm Hg in P(A-a)O₂ between these two pre-randomization time points: 18 (24.7%) had an increase in P(A-a)O₂ ≧5 mm Hg, while 13 (17.8%) had a decrease ≧5 mm Hg (Table 9). In contrast, none of 81 tested patients had a ≧10% decrease in % predicted FVC, and only 1 (1%) patient had a ≧10% increase in % predicted FVC.

TABLE 9
Change from the Screening Visit to the Baseline
Visit in P(A-a)O₂ and Percent Predicted FVC*

| | IFN-γ 1b | Placebo |
|---|---|---|
| | n = 36 | n = 37 |
| Change in P(A-a)O₂ | | |
| ≧5 mm Hg increase | 11 (30.6%) | 7 (18.9%) |
| ≧5 mm Hg decrease | 9 (25.0%) | 4 (10.8%) |
| | n = 41 | n = 40 |
| Change in % predicted FVC | | |
| ≧10% decrease | 0 (0%) | 0 (0%) |
| ≧10% increase | 0 (0%) | 1 (2.5%) |

*P(A-a)O₂ = alveolar-arterial oxygen gradient.

Relationship Between Change in Physiologic Parameters and Mortality

The relationships between the greatest change in P(A-a)O₂ and mortality, and between % predicted FVC and mortality were assessed in all placebo patients (Table 10). Change in P(A-a)O₂ was not associated with an increased risk of death in patients at increases of 1-14 mmr Hg, but mortality increased 2.4-fold in those who had increases of ≧15 mm Hg. In contrast, the protocol-defined threshold of ≧10% decrease in % predicted FVC was associated with a 2.4-fold increase in the relative risk of death.

The change from baseline in % predicted DLCO was also examined. There was no obvious threshold that was associated with a substantially increased risk of death (Table 10).

TABLE 10
Mortality According to Greatest Change in Physiologic
Parameters During the Study Period in Patients Receiving Placebo*

| | Total Number of | | |
|---|---|---|---|
| | Deaths/ | | |
| | Total Number of | | Relative Risk |
| | Patients (%) | | of Death |
| Change from baseline in† | | | |
| P(A-a)O ₂ | | | |
| No change or improvement | 3/22 | (14%) | 1.0 |
| 1-4 mm Hg increase | 4/38 | (11%) | 0.8 |
| 5-9 mm Hg increase | 0/27 | (0%) | NA |
| 10-14 mm Hg increase | 5/37 | (14%) | 1.0 |
| ≧15 mm Hg increase | 13/39 | (33%) | 2.4 |
| Missing‡ | 3/5 | (60%) | 4.3 |
| Change from baseline in % | | | |
| predicted FVC | | | |
| No change or improvement† | 3/24 | (13%) | 1.0 |
| 1-4% decrease | 1/41 | (2%) | 0.2 |
| 5-9% decrease | 6/49 | (12%) | 0.9 |
| ≧10% decrease | 15/49 | (31%) | 2.4 |
| Missing‡ | 3/5 | (60%) | 4.6 |
| Change from baseline in | | | |
| % predicted DLCO | | | |
| No change or improvement† | 5/26 | (19%) | 1.0 |
| 1-4% decrease | 2/48 | (4%) | 0.2 |
| 5-9% decrease | 9/44 | (20%) | 1.1 |
| 10-14% decrease | 3/23 | (13%) | 0.7 |
| ≧15% decrease | 5/23 | (22%) | 1.2 |
| Missing‡ | 4/6 | (67%) | 3.5 |

*P(A-a)O₂ = alveolar-arterial oxygen gradient; DLCO = diffusing capacity of the lung for carbon monoxide.
‡Reference group.
†Second measurement was not available for comparison to baseline in these patients.

Analyses to Assess the Impact of IFN-γ1b Treatment on Disease Progression and Mortality

Although analysis of the protocol-specified definition for the primary efficacy endpoint did not appear to reflect a treatment effect of IFN-γ 1b (p=0.53), a smaller proportion of IFN-γ 1b patients reached the endpoint than did placebo patients in every analysis that used any combination of components of the endpoint definition (Table 5). Similarly, patients receiving IFN-γ 1b had lower frequencies of every outcome in which the threshold for change in P(A-a)O₂ or % predicted FVC was varied, either in combination or as isolated parameters (Table 8). An intent-to-treat analysis identified a trend toward enhanced survival in patients receiving IFN-γ 1b, with death in 16 (9.9%) IFN-γ 1versus 28 (16.7%) placebo patients (p=0.08, log rank test). In every subcategory of baseline P(A-a)O₂, % predicted FVC, and % predicted DLCO composed of ≧5 deaths, the proportion of patients dying was similar to or lower in the IFN-γ 1b group than in the placebo group (Table 11). Similarly, an analysis in which dichotomized subgroups of these 3 baseline variables were assessed in association with mortality showed a lower proportion of deaths in the IFN-γ 1b group than in the placebo group in every analysis composed of ≧5 events.

TABLE 11
Mortality According to Treatment Group and
Baseline Physiologic Characteristic*

| | Total Number of Deaths/ | | | |
|---|---|---|---|---|
| | Total Number of Patients (%) | | | |
| | IFN-γ 1b | | Placebo | |
| Baseline P(A-a)O₂ (mm Hg) | | | | |
| Categorical subgroups | | | | |
| <10 | 1/16 | (6.3%) | 1/21 | (4.8%) |
| 10-19 | 2/31 | (6.5%) | 1/32 | (3.1%) |
| 20-29 | 5/56 | (8.9%) | 9/59 | (15.3%) |
| 30-39 | 3/43 | (7.0%) | 10/42 | (23.8%) |
| 40-49 | 4/13 | (30.8%) | 7/14 | (50.0%) |
| 50-59 | 1/2 | (50%) | 0/0 | |
| Dichotomous subgroups | | | | |
| <10 | 1/16 | (6.3%) | 1/21 | (4.8%) |
| <20 | 3/47 | (6.4%) | 2/53 | (3.8%) |
| <30 | 8/103 | (7.8%) | 11/112 | (9.8%) |
| <40 | 11/146 | (7.5%) | 21/154 | (13.6%) |
| <50 | 15/159 | (9.4%) | 28/168 | (16.7%) |
| <60 | 16/161 | (9.9%)† | 28/168 | (16.7%) |
| Baseline % predicted FVC | | | | |
| Categorical subgroups | | | | |
| ≧90 | 0/1 | (0%) | 1/3 | (33.3%) |
| 80-89 | 0/14 | (0%) | 2/16 | (12.5%) |
| 70-79 | 3/31 | (9.7%) | 6/33 | (18.2%) |
| 60-69 | 1/52 | (1.9%) | 5/44 | (11.4%) |
| 50-59 | 12/59 | (20.3%) | 12/65 | (18.5%) |
| 40-49 | 0/5 | 0% | 2/7 | (28.6%) |
| Dichotomous subgroups | | | | |
| ≧70 | 3/46 | (6.5%) | 9/52 | (17.3%) |
| ≧65 | 3/67 | (4.5%) | 10/77 | (13.0%) |
| ≧60 | 4/98 | (4.1%) | 14/96 | (14.6%) |
| ≧55 | 6/126 | (4.8%) | 21/128 | (16.4%) |
| ≧50 | 16/157 | (10.2%) | 26/161 | (16.1%) |
| ≧45 | 16/160 | (10.0%) | 28/168 | (16.7%) |
| ≧40 | 16/162 | (9.9%) | 28/168 | (16.7%) |
| Baseline % predicted DLCO | | | | |
| Categorical subgroups | | | | |
| ≧50 | 0/15 | (0%) | 1/23 | (4.3%) |
| 40-49 | 3/42 | (7.1%) | 3/31 | (9.7%) |
| 30-39 | 1/60 | (1.7%) | 12/67 | (17.9%) |
| 20-29 | 11/44 | (25.0%) | 11/44 | (25.0%) |
| 10-20 | 1/1 | (100%) | 1/3 | (33.3%) |
| Dichotomous subgroups | | | | |
| ≧50 | 0/15 | (0%) | 1/23 | (4.3%) |
| ≧45 | 2/33 | (6.1%) | 1/37 | (2.7%) |
| ≧40 | 3/57 | (5.3%) | 4/54 | (7.4%) |
| ≧35 | 4/87 | (4.6%) | 11/85 | (12.9%) |
| ≧30 | 4/117 | (3.4%) | 16/121 | (13.2%) |
| ≧25 | 12/152 | (7.9%) | 25/158 | (15.8%) |
| ≧20 | 15/161 | (9.3%) | 27/165 | (16.4%) |
| ≧15 | 16/162 | (9.9%) | 28/168 | (16.7%) |

*P(A-a)O₂ = alveolar-arterial oxygen gradient; DLCO = diffusing capacity of the lung for carbon monoxide.
†Measurement of P(A-a)O₂ at baseline was not performed in one study patient.

The mortality endpoint was more sensitive to a treatment effect of IFN-γ 1b than physiologic markers of disease progression. In the mortality analysis, there were two subgroups of patients in which the evidence for a treatment effect was strongest, in terms of both magnitude of effect and sensitivity to treatment: those with baseline % predicted FVC ≧55% (death in 4.8% IFN-γ 1b versus 16.4% placebo patients; 71% relative reduction in the risk of death; p=0.004, log rank test) and those with baseline % predicted DLCO ≧30% (death in 3.4% IFN-γ 1b versus 13.2% placebo patients; 74% relative reduction in the risk of death; p=0.008). Of note is that each of these two subgroups included a majority of study patients (254 [77.0%] patients had baseline % predicted FVC ≧55% and 238 [72.1%] patients had baseline % predicted DLC ≧30%).

### Example 3

### Effect of IFN-γ1b on TGF-β-Induced ECM Accumulation

IPF is a crippling disease that impairs gas exchange in the lung due to excessive accumulation of extra cellular matrix (ECM). IPF is thought to result from epithelial cell injury followed by aberrant wound healing. Numerous resident and recruited cell types, including lung epithelial cells, fibroblasts, activated macrophages, platelets, and lymphocytes, are known to release transforming growth factor beta (TGF-beta) in lung tissue in individuals with IPF.

TGF-beta, in turn, enhances the deposition and accumulation of ECM, which leads to fibrotic lesions. To examine the molecular consequences of therapeutic application of IFN-gamma 1b, the effect of IFN-gamma 1b on TGF-beta-modulated ECM turnover was studied in a cellular model of IPF.

Methods

A human lung epithelial cell line (A549) was cultured in DMEM culture medium containing 10% serum, then washed with phosphate-buffered saline (PBS); afterwards, serum-free medium was added to the cells. After overnight incubation in the serum-free medium, cells were treated with increasing concentrations of IFN-gamma 1b, or left untreated, and then stimulated with 5 ng/ml TGF-beta. Both cell culture supernatant and cell lysate were collected, and enzyme-linked immunosorbent assay (ELISA) was used to quantify secreted collagen and intracellular tissue inhibitor of metalloproteases 1 (TIMP1).

Results

Relative to untreated cells, TGF-beta induced the expression of collagen by 30% and TIMP1 by 60%. TGF-beta-induced expression of both collagen and TIMP1 was suppressed in a concentration-dependent fashion by addition of IFN-gammalb (p 0.01 for TIMP1 and 0.03 for collagen). Importantly, these effects were statistically significant at the therapeutically relevant Cmax concentrations of IFN-gammalb obtained from clinical trials.

These results indicate that IFN-gammalb inhibits both TGF-beta-induced collagen synthesis and TGF-beta-induced accumulation of TIMP1. Since both of these components are integral to deposition and accumulation of extracellular matrix (ECM), the likely net result may be a substantial decrease in the rate of ECM accumulation. These results suggest that IFN-gammalb may be beneficial in the treatment for IPF in certain patients by reducing the rate of TGF-beta-induced ECM accumulation.

### REFERENCES

1. Schwartz D A, Helmers R A, Galvin J R, et al. Determinants of survival in idiopathic pulnonary fibrosis. Am J Respir Crit Care Med 1994; 149:450-4.
2. Bjoraker J A, Ryu J H, Edwin M K, et al. Prognostic significance of histopathologic subsets in idiopathic pulmonary fibrosis. American Journal of Respiratory Critical Care Medicine 1998; 157:199-203.
3. Douglas W W, Ryu J H, Schroeder D R. Idiopathic pulmonary fibrosis: Impact of oxygen and colchicine, prednisone, or no therapy on survival. Am J Respir Crit Care Med 2000;161:1172-8.
4. American Thoracic Society. Idiopathic pulmonary fibrosis: diagnosis and treatment. International consensus statement. Am J Respir Crit Care Med 2000; 161:646-664.
5. Johnson M A, Kwan S, Snell N J C, Nunn A J, Darbyshire J H, Turner-Warwick M. Randomised controlled trial comparing prednisolone alone with cyclophosphamide and low dose prednisolone in combination in cryptogenic fibrosing alveolitis. Thorax 1989; 44:280-8.
6. Raghu G, Depaso W J, Cain K, et al. Azathioprine combined with prednisone in the treatment of idiopathic pulmonary fibrosis: a prospective double-blind, randomized, placebo-controlled clinical trial. Am Rev Respir Dis 1991; 144:291-6.
7. van Oortegem K, Wallaert B, Marquette C H, et al. Determinants of response to immunosuppressive therapy in idiopathic pulmonary fibrosis. Eur Respir J 1994; 7:1950-7.
8. Selman M, Carrillo G, Salas J, et al. Colchicine, D-penicillamine, and prednisone in the treatment of idiopathic pulmonary fibrosis: a controlled clinical trial. Chest 1998; 114:507-12.
9. Flaherty K R, Toews G B, Lynch J P III, et al. Steroids in idiopathic pulmonary fibrosis: a prospective assessment of adverse reactions, response to therapy, and survival. Am J Med 2001; 110:278-82.
10. Billiau A., Vanderbroeck K. IFN γ. In: Cytokine Reference. A compendium of cytokines and other mediators of host defense. Oppenheim J J, Feldmann M, eds. 2001, Academic Press: pp. 641-688.
11. Narayanan A S, Whithey J, Souza A, Raghu G. Effect of gamma-interferon on collagen synthesis by normal and fibrotic human lung fibroblasts. Chest 1992; 101:1326-31.
12. Clark J G, Dedon T F, Wayner E A, Carter W G. Effects of interferon-gamma on expression of cell surface receptors for collagen and deposition of newly synthesized collagen by cultured human lung fibroblasts. J Clin Invest 1989; 83:1505-11.
13. Okada T, Sugie I, Aisaka K. Effects of gamma-interferon on collagen and histamine content in bleomycin-induced lung fibrosis in rats. Lymphokine Cytokine Res 1993; 12:87-91.
14. Lack G, Gelfand E W. The role of nebulized IFN-gamma in the modulation of allergic responses. Adv.Exp Med Biol 1996;409:17-23.
15. Gajewski T F, Fitch F W. Anti-proliferative effect of IFN-gamma in immune regulation. I. IFN-gamma inhibits the proliferation of Th2 but not Th1 murine helper T lymphocyte clones. J Immunol 1998;140:4245-5252.
16. Neumann B, Emmanulidis K, Stadler M, Holzmann B. Distinct function of interferon-gamma for chemokine expression in models of acute lung inflammation. Immunology 1998;95:512-521.
17. Ziesche R, Hofbauer E, Wittmann K, Petkov V, Block L-H. A prelinary study of long-term treatment with interferon gamma-lb and low-dose prednisolone in patients with idiopathic pulmonary fibrosis. N Engl J Med 1999; 341:1264-9.
18. Ziesche R, Bradford W Z, Crager M, Block L-H. Long-term survival in idiopathic pulmonary fibrosis patients treated with interferon gamma-1b (abstract). Chest 2002; 122: 75S-76S.
19. Chang J A, Curtis J R, Patrick D L, Raghu G. Assessment of health-related quality of life in patients with interstitial lung disease. Chest 1999; 116:1175-82.
20. Mahler D A, Weinberg D H, Wells C K, Feinstein A R. The measurement of dyspnea Contents, interobserver agreement, and physiologic correlated of two new clinical indexes. Chest 1984; 85:751-758.
20a. King T E, Jr., Tooze J A, Schwarz M I, Brown K, Cherniack R M. Predicting survival in idiopathic pulmonary fibrosis scoring system and survival model. Am J Respir Crit Care Med 2001; 164:1171-1181.
20b. Panos R J, Mortenson R L, Niccoli S A, King T E Jr. Clinical deterioration in patients with idiopathic pulmonary fibrosis: causes and assessment. Am J Med 1990; 88(4):396-404
21. Keogh BA, Bernardo J, Hunninghake G W, Line B R, Price D L, Crystal R G. Effect of intermittent high dose parenteral corticosteroids on the alveolitis of idiopathic pulmonary fibrosis. Am Rev Respir Dis 1983; 127:18-22.
22. Douglas W W, Ryu J H, Swensen S J et al. Colchicine versus prednisone in the treatment of idiopathic pulmonary fibrosis. A randomized prospective study. Am J Respir Crit Care Med 1998; 158:220-5.
23. O'Dormell K, Keogh B, Cantin A, Crystal R G. Pharmacologic suppression of the neutrophil component of the alveolitis in idiopathic pulmonary fibrosis. Am Rev Respir Dis 1987; 136:288-92.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a patient suffering from idiopathic pulmonary fibrosis, comprising administering to the patient an effective amount of IFN-γ, where the patient has a forced vital capacity (FVC) that is at least about 55% of the predicted normal value and/or has a carbon monoxide diffusing capacity (DL_{CO}) that is at least about 30% of the predicted normal value.

2. A method of increasing the probability of survival of a patient suffering from idiopathic pulmonary fibrosis (IPF), comprising administering to the patient an effective amount of IFN-γ, where the patient has a forced vital capacity (FVC) that is at least about 55% of the predicted normal value and/or has a carbon monoxide diffusing capacity (DL_{CO}) that is at least about 35% of the predicted normal value.

3-4. (canceled)

5. The method of claim 1, wherein the patient has a DL_{CO} that is at least 35% of the predicted normal value.

6. The method of claim 1, wherein IFN-γ is administered to the patient for a period selected from about 48 weeks. about 60 weeks, about one year, about 70 weeks, about 93 weeks, about 2 years, and the remainder of the patient's life.

7-12. (canceled)

13. The method of claim 1, wherein the method further comprises administering a corticosteroid to the individual.

14. The method of claim 1, wherein IFN-γ is administered in a dose of from about 1.48 μg/kg body weight to about 4.4 μg/kg body weight.

15. The method of claim 1, wherein IFN-γ is administered in a dose of about 200 μg.

16. The method of claim 1, wherein IFN-γ is administered three times weekly.

17. The method of claim 1, wherein IFN-γ is administered by subcutaneous administration.

18-44. (canceled)

45. The method of claim 2, wherein IFN-γ is administered to the patient for a period selected from about 48 weeks, about 60 weeks, about one year, about 70 weeks, about 93 weeks, about 2 years, and the remainder of the patient's life.

46. The method of claim 2, wherein the method further comprises administering a corticosteroid to the individual.

47. The method of claim 2, wherein IFN-,y is administered in a dose of from about 1.48 μg/kg body weight to about 4.4 μg/kg body weight.

48. The method of claim 2, wherein IFN-γ is administered in a dose of about 200 μg.

49. The method of claim 2, wherein IFN-γ is administered three times weekly.

50. The method of claim 2, wherein IFN-γ is administered by subcutaneous administration.
